(19) United States
(12) Patent Application Publication
(10) Pub. No.: US 2007/0292497 A1
(43) Pub. Date: Dec. 20, 2007

(54) Method for treating micrometastatic tumors

(76) Inventor: Francis J. Martin, San Francisco, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/799,652

(22) Filed: May 2, 2007

Related U.S. Application Data
(60) Provisional application No. 60/798,424, filed on May 4, 2006.

(51) Int. Cl. A61K 31/70; A61K 35/00; A61K 9/127; A61P 35/00

(52) U.S. Cl. 424/450; 424/93.1; 514/34

(57) ABSTRACT

An adjuvant chemotherapeutic method for treatment of micrometastases is described. The method comprises administering, to a subject previously treated for resection or reduction of a primary tumor, a chemotherapeutic agent entrapped in liposomes, the liposomes having a coating of hydrophilic polymer chains and a targeting ligand that specifically binds to micrometastases, for delivery of the chemotherapeutic agent to the micrometastases.

### CROSS REFERENCE TO RELATED U.S. APPLICATION DATA

The present application is derived from and claims priority to provisional application U.S. Ser. No. 60/798,424, filed May 4, 2006, which is herein incorporated by reference in its entirety.

### TECHNICAL FIELD

The subject matter described herein relates to a method for treating micrometastatic tumors residing in otherwise normal non-cancerous tissue. More particularly, the subject matter relates to an adjuvant chemotherapeutic method for treating micrometastatic tumors.

### BACKGROUND

A primary reason for morbidity and mortality in cancer patients is the development of metastatic lesions that progress, invade vital organs, and eventually lead to organ failure. Metastatic lesions develop in healthy non-cancerous tissues and organs as the result of migration and invasion of cancer cells originating from the primary tumor. Subsequent to tissue invasion, such metastatic cells, which have adapted to proliferate in these sites, undergo multiple rounds of mitosis forming micrometastatic lesions comprised of a collection of cancer cells that initially derive nutrients from and exchange gasses with the local environment. Once such micrometastastes have reached a threshold size of usually greater than a few cubic millimeters neovascularization is required to provide nutrients and to support further disease progression (Reijnveld J. C. et al., J. Neurol., 247(8):597-608 (2000); Ellegala, D. B. et al., Circulation, 108(3):336-41 (2003)).

It is recognized that the vasculature of established tumors is defective, exhibiting gaps in the vascular endothelium and having a poorly formed or no basement membrane (Dvorak, H. F. et al., Am. J. Pathol. 133(1):95-109 (1998); Hobbs, S. K. et al., Proc. Natl. Acad. Sci. U.S.A., 95(8):4607-12 (1998); Jain, R. K. Adv. Drug. Deliv. Rev., 46(1-3):149-68 (2001)). These gaps in the endothelium have been exploited to serve as a route of entry for colloidal drug delivery systems, such as pegylated liposomes, administered intravenously. Such liposomes can extravasate through the defective vasculature network supplying a tumor leading to “passive” tumor targeting, greater deposition of drug to the tumor site, and improved therapy (Allen T. A. et al., Semin. Oncol., 31(6 Suppl 13):5-15 (2004); Vail, D. M. et al., Semin. Oncol., 31(6 Suppl 13): 16-35 (2004)).

Therapeutic improvements using this approach are dependent upon the vascular tumor endothelium serving as the rate-limiting portal of entry for the liposomes into the tumor. Evidence supporting improved therapy in animal models and human disease has shown that a development of such leaky neovasculature in tumors is a prerequisite for improved therapy (Allen, supra; Vail, supra). Indeed, the lack of pathological vascularity in tumors has been proposed to explain the lack of therapeutic improvement by colloidal drug delivery systems when such systems are used to treat micrometastatic lesions that have not yet developed a vascular supply.

Thus, such non-vascularized micrometastatic lesions in tissues evade the therapeutic improvement offered by such colloidal drug delivery systems that rely upon the so-called “enhanced permeability and retention” (EPR) effect for their clinical utility. The EPR effect requires that the drug-carrying particle (such as a liposome or microcapsule) be small enough to pass through the defects in the tumor vasculature, i.e., extravasate (Maeda, H. Adv. Enzyme Regul., 41:189-207 (2001)). Following tumor entry, the particles become trapped in the tumor interstitium, often in close proximity to the site through which they extravasated, forming a focal perivascular pattern. The particles are not cleared by lymphatic drainage, as lymphatic drainage is compromised or non-existent in tumors. The particles release their entrapped drug, which subsequently enters nearby tumor cells and exerts a cytotoxic action.

In contrast to tumors that have developed their own vascular supply, micrometastases, also referred to as isolated disseminated tumor cells, that have invaded and reside in a normal, healthy tissue site have no vascular supply, but derive nutrients and exchange gasses similarly to the normal tissue cells surrounding them. Thus there is no anatomical access for a colloidal drug particle to reach such cells. Even in the event that the colloidal particle were to extravasate through normal blood vessels supplying the tissue, the particles would be cleared from the site by draining lymphatics and thus would not have an opportunity to release their drug in close proximity to the tumor cells. Thus, such micrometastatic cells/lesions escape therapy.

The evidence is overwhelming that adjuvant chemotherapy with conventional single agents, such as anthracyclines, and combinations of agents, such as doxorubicin plus cyclophosphamide and doxorubicin plus a taxane, produces a highly statistically significant reduction in the annual odds of cancer recurrence and death from cancers, such as breast cancer (Abrams, J. S., Breast Cancer, 8(4):298-304 (2001)). In the adjuvant setting, patients are treated with chemotherapy empirically following resection of the primary tumor. There is usually no objective evidence of active or measurable disease at the time chemotherapy is administered, but on the basis of staging criteria and prognostic indicators, treatment is initiated on the presumption that tumors cells have metastasized to distant sites, invaded normal tissues, and have a high likelihood of progressing into a metastatic lesion. Conventional chemotherapeutic agents normally used in the adjuvant setting cannot penetrate into such micrometastatic lesions when encapsulated in colloidal drug delivery systems such as pegylated liposomes since there is no compromised vasculature to serve as a route of entry for the drug-delivery particles into the tumor. Thus, such drugs encapsulated in colloidal systems have not provided a benefit to patients in the adjuvant setting.

Actively targeted, ligand-bearing, liposomes have been reported to improve therapy relative to untargeted liposomes in animal tumor models, provided that the target tumor cells express the appropriate receptor, e.g., one that binds the ligand and, preferably, provokes internalization of the drug-loaded liposome (Park, J. W. et al., Semin. Oncol., 31(6 Suppl 13):196-205 (2004)). However, all evidence presented thus far suggests that such ligand-bearing liposomes must also enter tumors through the compromised vascular endothelium, after such vessels have developed in the tumor (Maruyama, K. et al., Adv. Drug Deliv. Rev., 40(1-2):89-102 (1999); Park, J. W., Breast Cancer Res., 4(3):95-9 (2002)). This understanding has lead to the belief that both passively and actively targeted liposomes have limited therapeutic effect in the treatment of micrometastatic disease prior to neovascularization.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, an adjuvant chemotherapeutic method is provided. The method comprises administering, to a subject previously treated for resection or reduction of a primary tumor, a chemotherapeutic agent entrapped in liposomes, the liposomes having a coating of hydrophilic polymer chains and a targeting ligand that specifically binds to micrometastases, for delivery of the chemotherapeutic agent to the micrometastases.

In one embodiment, the method is for use in a subject previously treated for a breast cancer tumor.

In another embodiment, the liposomes have a targeting ligand for a HER2 receptor or for a growth factor receptor, such as the epidermal growth factor receptor. The targeting ligand is selected, in one embodiment, for specific binding with cancer cells that express the epidermal growth factor receptor, such as non-small cell lung cancer, colorectal cancer, or bladder cancer.

In another embodiment, the liposomes have an outer surface coating of the hydrophilic polymer poly(ethylene glycol). In still another embodiment, the liposomes have a diameter of between about 50-250 nm, more preferably of between about 50-100 nm.

The entrapped chemotherapeutic agent, in another embodiment, is an anthracycline.

In another aspect, a treatment method comprised of selecting a patient at risk of developing disseminated tumor cells or identified as having disseminated tumor cells, and administering a chemotherapeutic agent entrapped in liposomes, the liposomes having a coating of hydrophilic polymer chains and a targeting ligand that specifically binds to micrometastases.

In one embodiment, the selected patient is one at risk of developing disseminated tumor cells based on diagnosis of a cancer capable of metastasizing.

In another embodiment, the selected patient is one previously treated by surgical resection of a primary tumor. In exemplary embodiments, the patient is one previously treated for breast cancer, non-small cell lung cancer, colorectal cancer, prostate cancer, or bladder cancer.

In another embodiment, the selected patient is one previously treated with radiation therapy for reduction of a primary tumor.

In still another aspect, a method for treating micrometastases is provided. The method comprises administering, to a subject previously treated for resection or reduction of a primary tumor, a chemotherapeutic agent entrapped in liposomes, liposomes having a coating of hydrophilic polymer chains and a targeting ligand that specifically binds to micrometastases, for delivery of the chemotherapeutic agent to the micrometastases.

In one embodiment, the liposomes comprise a ligand that is internalized by a receptor on the micrometastases.

In addition to the exemplary aspects and embodiments described above, further aspects and embodiments will become apparent by reference to the sequences and by study of the following descriptions.

### BRIEF DESCRIPTION OF THE SEQUENCES

SEQ ID NO:1 is the nucleotide sequence of an antibody having binding affinity for the extracellular domain of c-erb-B2 receptor, also referred to herein as the HER2 receptor and the p185^{HER2} receptor.

SEQ ID NO:2 is the amino acid sequence of an antibody having binding affinity for the extracellular domain of c-erb-B2 receptor, also referred to herein as the HER2 receptor and the p185^{HER2} receptor. The antibody is referred to herein as a single chain antibody fragment, scFv.

### DETAILED DESCRIPTION

### I. Definitions

“Metastasis” refers to the transfer of malignant a tumor cell from one location to another. In one embodiment, metastasis refers to the transfer of a malignant tumor cell from one organ or tissue to another organ or tissue not directly connected with the first.

“Micrometastastes” refers to one or more isolated tumor cells that have undergone metastasis but lack a supporting vasculature. A micrometastastes when comprised of a single cell is also referred to herein as a disseminated isolated tumor cell.

Unless otherwise noted, the term “vesicle-forming lipid” refers to any lipid capable of forming part of a stable micelle or liposome composition and typically including one or two hydrophobic, hydrocarbon chains or a steroid group and may contain a chemically reactive group, such as an amine, acid, ester, aldehyde or alcohol, at its polar head group.

A “hydrophilic polymer” intends a polymer having some amount of solubility in water at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, peptidomimetics, and hydrophilic peptide sequences. The polymers may be employed as homopolymers or as block or random copolymers. A preferred hydrophilic polymer chain is polyethyleneglycol (PEG), preferably as a PEG chain having a molecular weight between 500-10,000 daltons, more preferably between 750-10,000 daltons, still more preferably between 750-5000 daltons.

As used herein, an “antibody” includes whole antibodies and any antigen binding fragment or single chain fragment thereof. Thus the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian HER1, HER2, HER3, and HER4 growth factor receptor. Examples of binding fragments encompassed within the term “antigen binding portion” of an antibody include (i) a Fab fragment, a monovalent fragment consisting of the VL, VH, CL and CH, domains; (ii) a F(ab′)₂ fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., Nature, 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science, 242:423-426(1988), Huston et al., Proc. Natl. Acad. Sci. USA, 85:5879-5883 (1988)). Such single chain antibodies are also intended to be encompassed within the term antibody and with “antigen-binding portion” of an antibody. These antibody fragments are obtained using conventional techniques known to those with skill in the art, and the fragments are screened for utility in the same manner as are intact antibodies.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

An “internalizing antibody” is an antibody that, upon binding to a receptor or other ligand on a cell surface, is transported into the cell, for example, into a vacuole or other organelle or into the cytoplasm.

As used herein, “specific binding” refers to antibody binding to a predetermined antigen.

The phrases “an antibody recognizing an antigen” and “an antibody specific for an antigen” are used interchangeably herein with the term “an antibody which binds specifically to an antigen”.

### II. METHOD OF TREATMENT

A treatment method is disclosed which provides improved therapy for microscopic “micrometastatic” tumors which have yet to elaborate a vascular supply of their own. Such tumors are quite small and can be a single cell or a collection of cells up to a few cubic millimeters in size. Micrometastatic tumors have invaded a normal, otherwise healthy tissue and cannot be treated effectively with drug-loaded colloidal delivery systems, such as polymer-coated liposomes, because no route of entry for the colloidal particle into the tissues exists prior to the development of neovascularization (i.e. angiogenesis). Thus, the EPR effect, which is the basis for the mechanism of action of such colloidal systems, is not applicable.

Liposomes having an outer surface coating of a hydrophilic polymer are capable of entering the tissue compartment in all major tissues and organs of the body through normal vessels and traffic through these tissues. When such liposomes bear an appropriate cell-specific ligand and encounter a cancer or tumor cell residing in a normal tissue expressing the appropriate cell surface receptor, the liposomes bind and are internalized by the tumor/cancer cell. A cytotoxic drug incorporated into the liposome is then effective to kill the tumor cell or collection of tumor cells. Thus, this method provides a treatment option to destroy micrometastatic tumor cells prior to their having developed an independent self-sustaining vascular supply.

A1. Liposome Components and Preparation

The liposomes for use in the method are preferably composed primarily of vesicle-forming lipids. Such a vesicle-forming lipid is one that can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes. The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids, cerebrosides and sterols, such as cholesterol.

The vesicle-forming lipid can be selected to achieve a specified degree of fluidity or rigidity, to control the stability of the liposome in serum, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. On the other hand, lipid fluidity is achieved by incorporation of a relatively fluid lipid, typically one having a lipid phase with a relatively low liquid to liquid-crystalline phase transition temperature, e.g., at or below room temperature.

The liposomes also preferably include a vesicle-forming lipid covalently linked to a hydrophilic polymer. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S., et al., Bioconjugate Chem. 8:111 (1997); Zalipsky, S., et al., Meth. Enzymol. 387:50, (2004). These lipopolymers can be prepared as well-defined, homogeneous materials of high purity, with minimal molecular weight dispersity (Zalipsky, S., et al., Bioconjugate Chem. 8:111, (1997); Wong, J., et al., Science 275:820, (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, for example, U.S. Pat. No. 5,013,556).

The lipopolymer is modified to include a ligand, forming a lipid-polymer-ligand conjugate, also referred to herein as a ‘lipopolymer-ligand conjugate’. It will be appreciated that more than one of the same or different ligand can be used. In a preferred embodiment, the ligand is a targeting molecule having binding affinity for a binding partner, preferably a binding partner on the surface of a cell. A preferred ligand has binding affinity for the surface of a cell and facilitates entry of the liposome into the cytoplasm of a cell via internalization. A ligand present in liposomes that include such a lipopolymer-ligand is oriented outwardly from the liposome surface, and therefore available for interaction with its cognate receptor. In embodiments where a second ligand is included in the liposome composition, the second ligand can be the same or a different targeting ligand, or can be a therapeutic molecule, such as a drug or a biological molecule having activity in vivo, or a diagnostic molecule, such as a contrast agent or a biological molecule.

Methods for attaching ligands to lipopolymers are known, where the polymer can be functionalized for subsequent reaction with a selected ligand (U.S. Pat. No. 6,180,134; Zalipsky, S. et al., FEBS Lett. 353:71 (1994); Zalipsky, S. et al., Bioconjugate Chem. 4:296 (1993); Zalipsky, S. et al., J. Control. Rel. 39:153 (1996); Zalipsky, S. et al., Bioconjugate Chem. 8(2):111 (1997); Zalipsky, S. et al., Meth. Enzymol. 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selectins, such as E, L, and P selectins, Flk-1,2, FGF, EGF, integrins, in particular, αᵥβ₄ αᵥβ₃, αᵥβ₁ αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388; 6,316,024; 6,056,973; 6,043,094, which are herein incorporated by reference.

In one embodiment, the ligand is one for an internalizing epitope. Selection of ligands that target tumor cells and undergo internalization is described for example in Gao, C. et al., J. Immunol. Methods, 274(102): 185 (2003). Examples of tumor-specific internalizing ligands include the single-chain antibody fragments identified herein as SEQ ID NOS:1-2. These scFv antibody ligands are specific for a HER2 cell receptor and suitable for treatment of breast cancer and other HER-2 overexpressing tumors. In another embodiment, an anti-EGFR targeting ligand is used for treatment of colorectal cancer and other EGFR-overexpressing tumors.

Preparation of liposomes is well known in the art, see, for example, Szoka, F. and Papahadjopoulos, D., Proc. Natl. Acad. Sci. USA 75:4194-4198, (1978) or Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)). Liposomes having a diameter of between about 50-150 nm, more preferably between about 50-100 nm, are preferred.

Incorporation of a lipid-polymer-ligand targeting conjugate is achieved by various approaches. One approach involves preparation of lipid vesicles that include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, for example, U.S. Pat. Nos. 6,224,903, 5,620,689). In yet another approach, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973, 6,316,024).

The liposomes can include an entrapped drug, such as a chemotherapeutic agent. There are no restrictions on the drug contemplated for use, and some exemplary preferred drugs include cytotoxics such anthracyclines, camptothecins, proteasome inhibitors, platinum compounds, vinca alkaloids, and kinase inhibitors.

A2. Adjuvant Treatment Method

In one embodiment, an adjuvant chemotherapeutic method is provided. In the method a subject previously diagnosed and treated for cancer is identified for treatment. The prior treatment is preferably treatment of a primary tumor by surgical resection and/or radiation therapy and/or chemotherapy. The prior treatment preferably achieved reduction in size of the primary tumor, either by surgical resection or by radiation or chemotherapy. As an adjuvant to treatment of the primary tumor, the subject is treated with a chemotherapeutic agent entrapped in liposomes as described above. The term “adjuvant chemotherapy” as used herein intends treatment using one or more anticancer drugs after initial treatment of a primary tumor, typically by surgery or radiotherapy, in a patient diagnosed with a cancer that is capable of metastasizing and/or likely to recur. In the adjuvant treatment method, the subject is treated with liposomes having a coating of hydrophilic polymer chains and a targeting ligand that specifically binds to micrometastases, for delivery of the chemotherapeutic agent to the micrometastases.

More generally, and in another embodiment, a method for treating micrometastases is provided. The method includes administering, to a subject previously treated for resection or reduction of a primary tumor, a chemotherapeutic agent entrapped in liposomes, the liposomes having a coating of hydrophilic polymer chains and a targeting ligand that specifically binds to micrometastases, for delivery of the chemotherapeutic agent to the micrometastases.

In another embodiment, a treatment method is provided that comprises selecting a patient at risk of developing disseminated tumor cells, at risk of having disseminated tumor cells, or identified as having disseminated tumor cells. The selected patient is treated by administering a chemotherapeutic agent entrapped in liposomes, the liposomes having a coating of hydrophilic polymer chains and a ligand for targeting to the disseminated tumor cells. Patients at risk of developing disseminated tumor cells include those that are diagnosed with a cancer capable of metastasizing, particularly if the diagnosis is made when the cancer is in an early stage. Patients at risk of having disseminated tumor cells include those diagnosed with cancer at any stage, particularly those diagnosed with a cancer known to metastasize and/or diagnosed with a late stage cancer.

Disseminated isolated tumor cells, or micrometastases, can be identified by cytopathology/histophathology, immunocytochemistry/immunohistochemistry, and polymerase chain reaction (PCR). In all the techniques, a sample from a subject is obtained, for example from a peritoneal wash, from bone marrow, or from the blood. Cytopathic or histopathologic dentification of isolated tumor cells in the sample is performed by staining the sample with, for example, Papanicolaou, the hematoxylin/eosin, or the Giemsa staining methods. The stained sample is evaluated by a pathologist or cytologist for the presence or absence of tumor cells. Detection of isolated tumor cells by immunohistochemistry uses monoclonal antibodies against an antigen of the tumor cell. For example, cytokeratins, an essential constituent of the cytoskeleton of all epithelial cells, can be used as a target on tumor cells in gastrointestinal tumors. CK2, an antibody directed against cytokeratin 18, is a specific example. PCR permits identification of disseminated tumor cells with high sensitivity and high specificity via detection of tumor-specific chromosomal rearrangement, detection of tumor-specific DNA mutations (such as k-ras or p53 mutations), or detection of a marker gene mRNA via reverse-transcriptase PCR.

Doses and a dosing regimen for the liposome formulation will depend on the cancer being treated, the chemotherapeutic agent entrapped in the liposomes, the stage of the cancer, the size and health of the patient, and other factors readily apparent to an attending medical caregiver.

The liposome formulation is typically administered parenterally, with intravenous administration preferred. It will be appreciated that the formulation can include any necessary or desirable pharmaceutical excipients to facilitate delivery.

From the foregoing, various aspects and embodiments illustrating the method are apparent. Heretofore it has been believed that the utility of both non-targeted and targeted liposome systems in treating solid tumors required the preexistence of leaky, compromised vasculature in the tumor as the route of entry for such systemically administered liposomes. Thus such systems were considered inappropriate for the treatment of micrometastatic disease prior to angiogenesis. The treatment method described herein extends the clinical utility of targeted systems to the adjuvant setting. The ability of ligand-targeted liposomes to extravasate through normal, non-cancerous tissue vasculature, albeit at a much slower rate than through the relatively defective vascular of tumors, provides a means to target micrometastases in these tissues. Such ligand-bearing liposomes become entrained within the flow of interstitial fluid in tissues and thus move from the sites of extravasation, through the tissue parenchyma and eventually enter and are drained from the tissues by efferent lymphatic vessels. During their transit through the tissue space, if an isolated disseminated tumor cell or collection of such cells is encountered, the liposomes interact and bind with such cells via the selected targeting ligand carried on the liposomes. In this way, micrometastatic cells which have invaded and taken up residence and begun to undergo mitosis in otherwise healthy tissue, but which have not yet provoked the development of their own vascular supply, are destroyed by the cytotoxic drug carried in the liposomes. It will be appreciated that a toxic ligand can be carried on the liposomes, and that a cytotoxic drug can be carried on the external surface of the liposomes. In this way, micrometastatic lesions can be eradicated, to reduce the chance of recurrence of cancer in the patient.

### III. EXAMPLES

The following examples are illustrative in nature and are in no way intended to be limiting.

### Example 1

### In vitro Binding of Targeted Liposomes to Tumor Cells

A. Preparation of Tumor Cell Line Overexpressing HER2 and GFP

BT-474 human breast cancer cells that overexpress HER2 are transfected with an expression vector containing the highly fluorescent S56T variant (GFP-S56T) of the wild-type green fluorescent protein (GFP) gene and a selectable marker. The cells are maintained at 37 C in Dulbecco's modified Eagle's medium (DMEM) supplemented with 10% fetal calf serum. The cells are routinely subcultured at a ratio of 1:10 in selective medium. Cells from confluent monolayers are harvested by trypsinization and resuspended in DMEM with fetal calf serum to a final concentration of 2×10⁶ cells/mL. Epi-fluorescence microscopy was used to verify membrane integrity by exclusion of ethidium-bromide. Stability of GFP fluorescence is characterized by growing the cells for 24 days, with a passage every fourth day. After each cell passage fluorescence intensity is assessed by flow cytometry.

B. Preparation of HER2-Targeted Immunoliposomes

Liposomes containing entrapped doxorubicin are obtained from Alza Corporation Mountain View, Calif. (DOXIL®). The liposomes are composed of hydrogenated soy phosphatidylcholine (HSPC, 56.4 mole %), cholesterol (38.3 mole %), and methoxypolyethyleneglycol-di-stearoyl-phosphatidylethanolamine (mPEG-DSPE, 5.3 mole %, mPEG MW 2000 Da). The concentration of doxorubicin in the final preparation is 100 μg/mM lipid. The internal buffer used for the preparation is 10% sucrose and the external buffer is 10% sucrose and 10 mM histidine. The average diameter of liposomes in the final formulation is 93 nm.

A single chain antibody (scFv) antibody (SEQ ID NO:2) is conjugated to a maleimide-derivatized PEGylated phospholipid (mPEG-DSPE) to form a lipid-PEG-scFv conjugate, according to procedures well known in the art. The lipid-PEG-anti-HER2 antibody construct was then associated with liposomal bilayers of the doxorubicin-loaded liposomes by incubation of the liposomes with a micellar suspension of the lipid-polymer-antibody construct. Immunoliposomes bearing on average 15 antibodies were prepared using 6 antibodies per mole of phospholipids. After association of the lipid-polymer-antibody conjugates with the liposomes, the doxorubicin concentration is 86-92 μg/mM lipid and the average diameter of immunoliposomes after is 93-117 nm.

C. In Vitro Uptake of Immunoliposomes

The transfected BT-474 cells at 1.5×10⁵ cells/0.5 mL of growth medium per well are added to a 24-well plate. After overnight incubation for attachment and acclimation, cells are treated with immunoliposomes or liposomes at 0.015 mg/mL in 0.5 mL growth media/well in duplicates. The 24-well plates are then placed on a rotating platform inside the incubator with rotation at 40-60 rpm at 37° C., 5% CO₂, and 100% humidity for 4 hours. After incubation, cell medium is aspirated and the cells are washed four times with Hank's Balanced Salt Solution (HBSS). After this, cells are lysed by adding 0.1 mL of 1% Triton X-100 to each well. The plates are swirled to mix and are placed on a rotating platform for 15 minutes at room temperature. During this step, cells detach from the bottom of the plate and the cell nuclei form visible clumps. Acid isopropanol (1.0 mL) is added to the wells containing the Triton X-100 solution and is mixed by swirling or pipetting up and down until the visible clumps disappear.

Doxorubicin content in the cell lysates is measured by a spectrofluorometer using an excitation wavelength of 470 nm and an emission wavelength of 590 nm. The spectrofluorometer settings are as follows: slit width 2 nm, integration time 1 second, photomultiplier voltage 950 V, and single wavelength acquisition mode. Fluorescence of the background cell lysate is subtracted and the amount of doxorubicin in the cell lysates is determined from concurrently measured standards (10, 25, 50, 100, 250, 500, 1000, and 2500 ng of doxorubicin per sample). Doxorubicin standard curve is fit using quadratic polynomial regression and cell uptake of doxorubicin is determined by interpolation.

### Example 2

### In vivo Binding of Targeted Liposomes to Disseminated Isolated Tumor Cells

NCr nu/nu female mice, 5-6 weeks old and 24-25 grams, are obtained. The mice are inoculated with BT474 tumor cells transfected with GFP as described in Example 1. One week after inoculation, mice are treated with immunoliposomes prepared as described in Example 1 or with liposomes lacking the targeting antibody (doxorubicin containing pegylated liposomes, DOXIL®). The liposomes are administered via tail vein injection at a doxorubicin dose of 2 mg/kg.

For three weeks post treatment, the body weight of the mice is monitored. Additionally, dual-color imaging is conducted on selected mice on days 1, 3, 6, 9, 12, 15, 18, and 21 to determine immunoliposome binding and uptake into individual non-dividing tumor cells.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. An adjuvant chemotherapeutic method, comprising:
administering, to a subject previously treated for resection or reduction of a primary tumor, a chemotherapeutic agent entrapped in liposomes, the liposomes having a coating of hydrophilic polymer chains and a targeting ligand that specifically binds to micrometastases, for delivery of the chemotherapeutic agent to the micrometastases.

2. The method of claim 1, wherein said administering comprises administering to a subject previously treated for a breast cancer tumor.

3. The method of claim 2, wherein said administering comprises administering liposomes having a targeting ligand for a HER2 receptor.

4. The method of claim 1, wherein said administering comprises administering liposomes having a targeting ligand for a growth factor receptor.

5. The method of claim 4, wherein said ligand binds to the epidermal growth factor receptor.

6. The method of claim 5, wherein said administering comprises administering to a subject previously treated for cancer comprised of cells that express the epidermal growth factor receptor.

7. The method of claim 6, wherein said administering comprises administering to a subject previously treated for non-small cell lung cancer, colorectal cancer, or bladder cancer.

8. The method of claim 1, wherein said administering comprises administering liposomes having the hydrophilic polymer poly(ethylene glycol).

9. The method of claim 1, wherein said administering comprises administering liposomes having a size of between about 50-100 nm.

10. The method of claim 1, wherein said administering comprises administering liposomes having an anthracycline as the entrapped chemotherapeutic agent.

11. A treatment method, comprising:
selecting a patient at risk of developing disseminated tumor cells or identified as having disseminated tumor cells;
administering a chemotherapeutic agent entrapped in liposomes, the liposomes having a coating of hydrophilic polymer chains and a targeting ligand that specifically binds to micrometastases.

12. The method of claim 11, wherein said selecting comprises selecting a patient at risk of developing disseminated tumor cells based on diagnosis of a cancer capable of metastasizing.

13. The method of claim 11, wherein said selecting comprises selecting a patient previously treated by surgical resection of a primary tumor.

14. The method of claim 13, wherein said selecting comprises selecting a patient previously treated for breast cancer.

15. The method of claim 14, wherein said administering comprises administering liposomes having a targeting ligand for a HER2 receptor.

16. The method of claim 12, wherein said selecting comprises selecting a patient previously treated for non-small cell lung cancer, colorectal cancer, prostate cancer, or bladder cancer.

17. The method of claim 16, wherein said administering comprises administering liposomes having a targeting ligand for the epidermal growth factor receptor.

18. The method of claim 11, wherein said selecting comprises selecting a patient previously treated with radiation therapy for reduction of a primary tumor.

19. A method for treating micrometastases, comprising
administering, to a subject previously treated for resection or reduction of a primary tumor, a chemotherapeutic agent entrapped in liposomes, liposomes having a coating of hydrophilic polymer chains and a targeting ligand that specifically binds to micrometastases, for delivery of the chemotherapeutic agent to the micrometastases.

20. The method of claim 19, wherein said administering comprises administering to a subject previously treated by surgical resection of a solid tumor.

21. The method of claim 19, wherein said administering comprises administering comprises administering liposomes comprising a ligand that is internalized by a receptor on the micrometastases.


SEQUENCE LISTING (2 sequences)

SEQ ID NO: 1 - protein, 738 aa, Homo sapiens
CAGGTGCAGCTGGTGGAGTCTGGGGGAGGCTTGGTACAGCCTGGGGGGTCCCTGAGACTCTCCTGTGCAGCCTCTGGATTCACCTTTCGCAGCTATGCCATGAGCTGGGTCCGCCAGGCTCCAGGGAAGGGGCTGGAGTGGGTCTCAGCTATTAGTGGTCGTGGTGATAACACATACTACGCAGACTCCGTGAAGGGCCGGTTCACCATCTCCAGAGACAATTCCAAGAACACGCTGTATCTGCAAATGAACAGCCTGAGAGCCGAGGACACGGCCGTTTATTACTGTGCGAAAATGACAAGTAACGCGTTCGCATTTGACTACTGGGGCCAGGGAACCCTGGTCACCGTCTCCTCAGGTGGAGGCGGTTCAGGCGGAGGTGGCTCTGGCGGTGGCGGATCGCAGTCTGTGTTGACGCAGCCGCCCTCAGTGTCTGGGGCCCCAGGGCAGAGGGTCACCATCTCCTGCACTGGGAGCAGCTCCAACATCGGGGCAGGTTATGGTGTACACTGGTACCAGCAGCTTCCAGGAACAGCCCCCAAACTCCTCATCTATGGTAACACCAATCGGCCCTCAGGGGTCCCTGACCGATTCTCTGGCTTCAAGTCTGGCACCTCAGCCTCCCTGGCCATCACTGGGCTCCAGGCTGAGGATGAGGCTGATTATTACTGCCAGTCCTATGACAGCAGCCTGAGTGGTTGGGTGTTCGGCGGAGGGACCAAGCTGACCGTCCTAGGT

SEQ ID NO: 2 - protein, 246 aa, Homo sapiens
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG